(19) United States
(12) Patent Application Publication
(10) Pub. No.: US 2013/0338616 A1
(43) Pub. Date: Dec. 19, 2013

(54) UROSTOMY FLUID DISCHARGE VALVE

(71) Applicant: Nu-Hope Laboratories, Inc., Pacoima, CA (US)

(72) Inventor: Bradley J. Galindo, Pacoima, CA (US)

(21) Appl. No.: 13/914,924

(22) Filed: Jun. 11, 2013

Related U.S. Application Data
(60) Provisional application No. 61/659,942, filed on Jun. 14, 2012.

(51) Int. Cl. A61F 5/44

(52) U.S. Cl. CPC A61F 5/4405
USPC 604/335

(57) ABSTRACT

A fluid discharge valve for releasing fluid from a urostomy pouch comprises a first valve element and a second valve element. The first valve element includes a first bore, a distal portion and a first flange element. The first flange element includes a multiplicity of first rotational detent elements. The second valve element includes a second bore, an upper portion, a lower portion and a flow conditioner within the second bore. The upper portion threadedly receives the distal portion for threaded movement of the second valve element between an open configuration and a closed configuration. The upper end includes a plurality of second rotational detent elements adapted to come into releasable engagement with respective first rotational detent elements when the second valve element is moved into its closed configuration, wherein the first bore is sealed shut. The releasable engagement inhibits movement of the second valve element from its closed configuration.

### RELATED APPLICATIONS

This application claims the benefit of U.S. Provisional Application No. 61/659,942, filed on Jun. 14, 2012, the content of which is incorporated by this reference in its entirety for all purposes as if fully set forth herein.

### TECHNICAL FIELD

The present invention relates generally to valve systems. More specifically, the present invention relates to actuatable discharge valves through which fluids may be drained from urostomy pouches.

### BACKGROUND

Urostomy pouches are used as a means to divert urine from a person's body in cases where drainage of urine through the bladder or urethra are not possible, such as after extensive bladder surgery or to bypass an obstruction. They may also be elected for use in cases of severe incontinence. When a urostomy pouch is employed, urine is passed out of the body through a stoma, and into the pouch. Urostomy pouches tend to be thin and watertight, and are typically designed to adhere to the skin of the user so that the user can carry on their daily lives while wearing the pouch. Urostomy pouches generally include a drain valve at the bottom of the pouch. Such drain valves may be opened and closed by way of simple actuation by the wearer. Among other concerns, common conventional urostomy drain valves may be vulnerable to inadvertently opening in response to movement by the wearer during their normal daily activities.

### SUMMARY

Certain deficiencies of the prior art may be overcome by the provision of a fluid discharge valve for releasing fluid from a urostomy pouch. Embodiments of such a fluid discharge valve comprise a first valve element and a second valve element.

The first valve element may include a proximal end, a distal end, a first bore extending therebetween, a proximal portion and a distal portion, a first axial detent element, and a first flange element disposed generally between the proximal and distal portions. The first flange element may include a first flange face and a multiplicity of first rotational detent elements. The proximal portion may be mated to an ostomy pouch having an internal fluid reservoir such that the first bore is in fluid communication with the internal fluid reservoir.

The second valve element may include an upper end, a lower end, a second bore extending therebetween, an upper portion, a lower portion, a second axial detent element, and a flow conditioner within the second bore. The flow conditioner may have a plug element and one or more vanes extending radially outwardly thereof. The upper portion may be adapted to threadedly receive at least part of the distal portion for threaded movement of the second valve element axially with respect to the first valve element between an open configuration and a closed configuration. The upper end may include a plurality of second rotational detent elements. The second rotational detent elements being adapted to come into releasable engagement with respective first rotational detent elements when the second valve element is moved into its closed configuration.

When the second valve element is in its closed configuration, the first bore may be sealed shut by the plug element. The releasable engagement inhibits threaded movement of the second valve element out of its closed configuration. This inhibition may be overcome by application of a greater torque on the second valve element than is required to effectuate substantially the remainder of the threaded movement. The first and second axial detent elements are adapted to engage one another to prevent the second valve element from axially separating from the first valve element when the second valve element is in its open configuration.

### BRIEF DESCRIPTION OF THE DRAWINGS

Further advantages of the present invention may become apparent to those skilled in the art with the benefit of the following detailed description of the preferred embodiments and upon reference to the accompanying drawings in which:

FIG. 1 is a diagrammatic partial view of an embodiment of a fluid discharge valve mated in fluid communication with an ostomy pouch;

FIG. 2 is a diagrammatic partial view of the embodiment of FIG. 1, but in which the second valve portion is shown in its open configuration and a discharge tube is shown connected to the lower portion of the second valve element;

FIG. 3 is a diagrammatic exploded view of an embodiment of a fluid discharge valve;

FIG. 4 is a further diagrammatic exploded view of the embodiment shown in FIG. 3;

FIG. 5 is a diagrammatic side view of an embodiment of a fluid discharge valve, wherein the second valve element is shown in a closed configuration;

FIG. 6 is a diagrammatic perspective view of the embodiment of FIG. 5;

FIG. 7 is a further perspective view of the embodiment of FIG. 5;

FIG. 8 is a diagrammatic side view of an embodiment of a fluid discharge valve, wherein the second valve element is shown in an open configuration;

FIG. 9 is a diagrammatic perspective view of the embodiment of FIG. 8;

FIG. 10 is a further perspective view of the embodiment of FIG. 8;

FIG. 11 is a diagrammatic end view of an embodiment of a fluid discharge valve;

FIG. 12 is a diagrammatic cross-sectional view taken along line 12-12 in FIG. 11, wherein the second valve element is in closed configuration, thereby preventing fluid from flowing from the first bore through the lower portion of the second valve element;

FIG. 13 is a diagrammatic cross-sectional view taken along line 13-13 in FIG. 11, wherein the second valve element is in closed configuration;

FIG. 14 is a diagrammatic cross-sectional view taken along line 12-12 in FIG. 11, wherein the second valve element is shown in open configuration, thereby allowing fluid to flow from the first bore through the lower portion of the second valve element;

FIG. 15 is a diagrammatic cross-sectional view similar to that of FIG. 14, but without the first valve element;

FIG. 16 is a diagrammatic partial broken out section view of a second valve element, illustrating a flow conditioner disposed within the second bore of the second valve element;

FIG. 17 is a diagrammatic partial broken out section view similar to that of FIG. 16, but illustrating the flow conditioner from a different angle;

FIG. 18 is a diagrammatic end view of an alternative embodiment of a fluid discharge valve, in which the flow conditioner has one vane;

FIG. 19 is a diagrammatic partial broken out section view of a second valve element in accordance with the embodiment shown in FIG. 18, illustrating a flow conditioner disposed within the second bore of the second valve element having, the second valve element having five rotational detent elements; and

FIG. 20 is a diagrammatic partial broken out section view similar to that of FIG. 19, but illustrating the flow conditioner from a different angle.

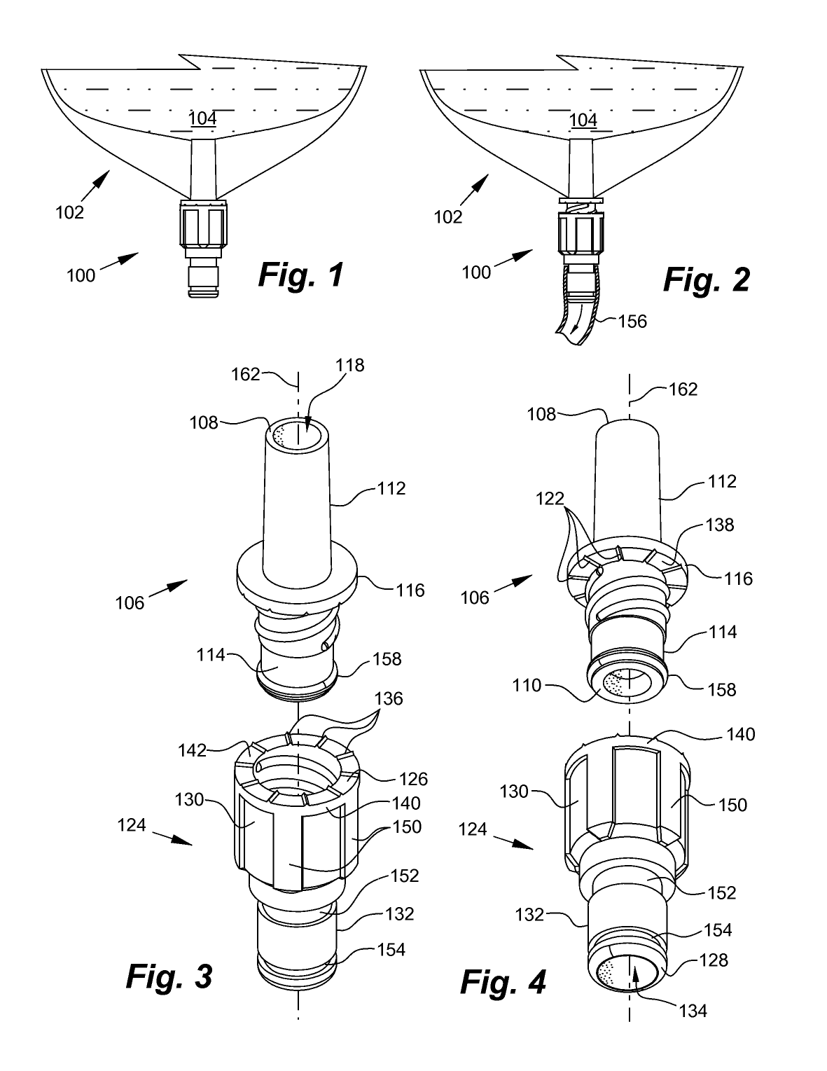

### DETAILED DESCRIPTION OF THE PREFERRED EMBODIMENTS

Referring now to the drawings, like reference numerals designate identical or corresponding features throughout the several views.

Embodiments of a fluid discharge valve are depicted 100, and may preferably be adapted as a connection interface for releasing fluid from a urostomy pouch. Embodiments of a fluid discharge valve 100 generally comprise a first valve element 106 and a second valve element 124 adapted to move threadably with respect to one another along a main axis 162.

A first valve element 106 may include a proximal end 108, a distal end 110, a first bore 118 extending therebetween and a first flange element 116. A proximal portion 112 may be adjacent the proximal end 108. A distal portion 114 may be adjacent the distal end 110. The first flange element 116 may be disposed generally between the proximal and distal portions, and may include at least one or a multiplicity of radially-distributed first rotational detent elements (individual examples of which are illustrated, for example, at 122). In certain preferred embodiments, the first flange element 116 may include a first flange face 138 being generally axially directed toward the distal portion 114. The first rotational detent elements 122 may be distributed along the first flange face 138.

A second valve element 124 may include an upper end 126, a lower end 128, a second bore 134 extending therebetween, an upper portion 130 and a lower portion 132. The lower portion 132 may be adjacent the lower end 128, and as illustrated for example in FIG. 2, is typically adapted to be removably sealingly received by a flexible tube or tubing 156. This feature generally allows a patient or wearer to conveniently connect the urostomy pouch 102 to a night drainage bag while sleeping. Securement of such connection may be facilitated, for example, by a first annular groove 152 and second annular groove 154 on the lower portion 132.

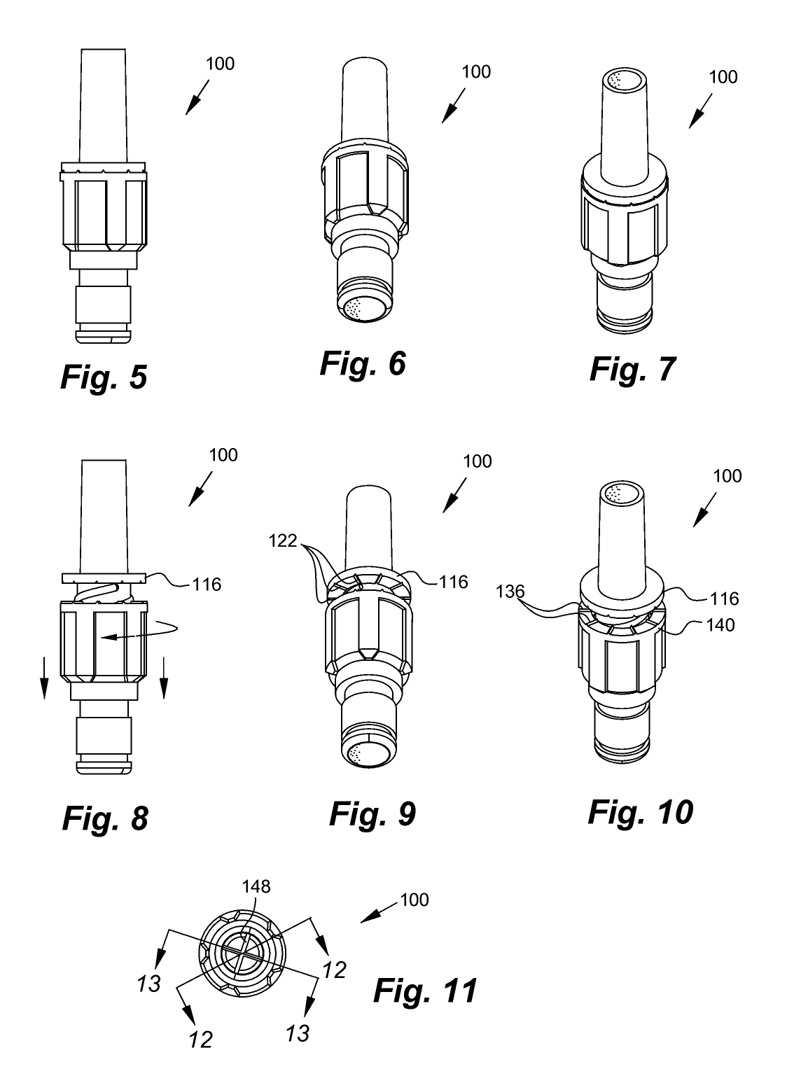

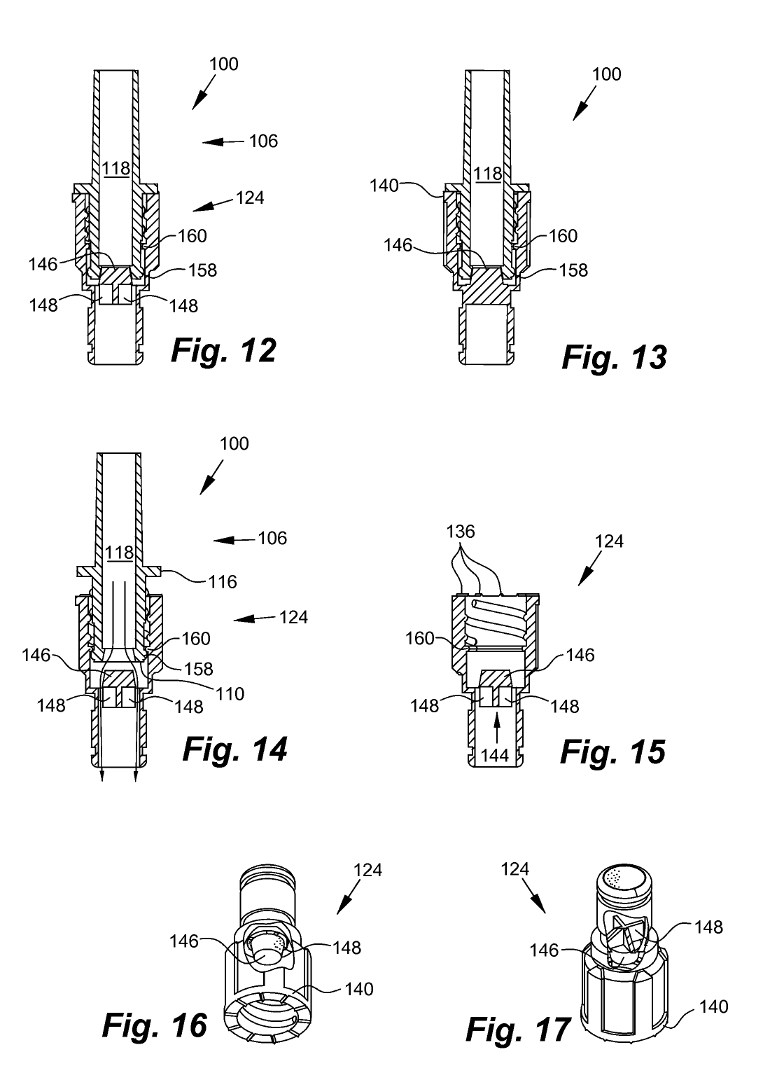

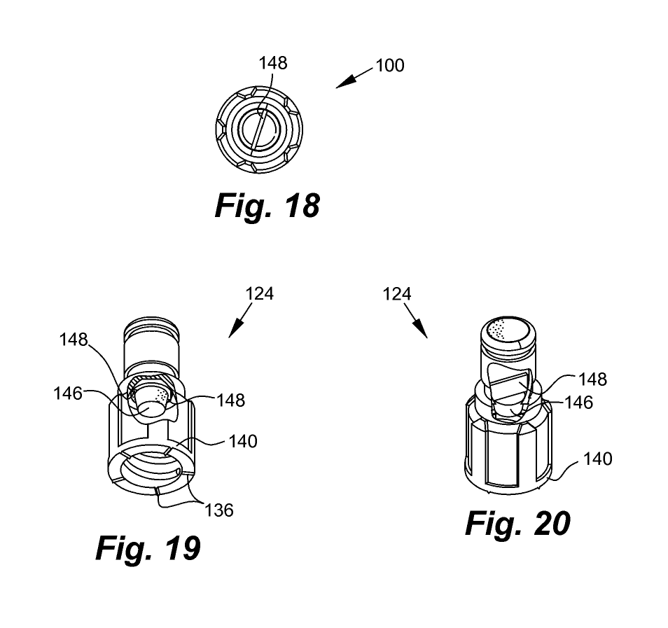

The upper portion 130 may be adjacent the upper end 126 and be adapted to threadedly receive at least a segment of the distal portion 114 for threaded movement of the second valve element 124 axially with respect to the first valve element 106 between an open configuration (see, for example, FIG. 14) and a closed configuration (see, for example, FIG. 12). The upper end 126 may preferably include at least one or a plurality of radially distributed second rotational detent elements (individual examples of which are illustrated, for example, at 136). As illustrated, for example, in FIGS. 5-7, the second rotational detent elements 136 are typically adapted to come into releasable engagement with respective first rotational detent elements 122 when the second valve element 124 is moved into its closed configuration. It should be noted that the terms “upper” and “lower” are used herein to differentiate various features and elements without necessarily limiting their respective orientation within the specification or claims.

The releasable engagement between first and second rotational detent elements typically inhibits threaded movement of the second valve element 124 out of its closed configuration. Thusly, in preferred embodiments, threaded movement of the second valve element 124 from its closed configuration toward its open configuration may present a “pop” or “snap” tactile effect to the user applying the requisite torque. A similar tactile effect may typically be experienced by the user upon final threaded movement of the second valve element 124 into its closed configuration. Such inhibition is generally intended to prevent unintentional movement of the second valve element 124 out of its closed configuration.

In preferred embodiments, this inhibition is adapted to be overcome by application of a greater torque or rotational force on the second valve element 124 than is generally required to effectuate substantially the remainder of the threaded movement. For example, if the threaded movement of the second valve element 124 requires a first torque throughout a majority of the movement between the closed and open configurations, the initial release of the second valve element 124 from its closed configuration may require the user to apply a second torque which is greater than the first torque. In certain such embodiments, a third torque may be required to return the second valve element 124 into its closed configuration, wherein the third torque may also be greater than the first torque. This third torque may be adapted to overcome the frictional and compressive resistance generated, for example, between the first flange element 116 and the second valve element 124 just prior to the releasable engagement between the first and second rotational detent elements.

Referring to FIGS. 12 and 13, for example, in certain preferred embodiments, the first bore 118 may be sealed shut when the second valve element 124 is in its closed configuration. Moreover, the second valve element 124 may include a flow conditioner 144 within the second bore 134. The flow conditioner 144 may reduce turbulence in the discharge fluid stream, thus facilitating faster drainage of the reservoir 104. The flow conditioner 144 may have a plug element 146 and one or more vanes 148 extending radially outwardly thereof. In such embodiments with a flow conditioner, for example, the first bore 118 may be adapted to be sealed shut by the plug element 146 when the second valve element 124 is in its closed configuration.

Referring to FIGS. 12 and 14 for illustration, in particular embodiments, the first valve element 106 may include a first axial detent element 158 and the second valve element 124 may include a second axial detent element 160. Referring more specifically to FIG. 14 for illustration, the first and second axial detent elements may be adapted to engage one another to prevent the second valve element 124 from axially separating from the first valve element 106 when the second valve element 124 is in its open configuration. Upon initial assembly of certain embodiments of the valve 100, the first axial detent element 158 may be forced axially past the second axial detent element 160 for securement of the first axial detent element 158 within the upper portion 130 of the valve 100.

As illustrated for example in FIGS. 3 and 4, in particular embodiments, the first rotational detent elements 122 and second rotational detent elements 136 may be radially distributed in a substantially even fashion. Moreover, in certain embodiments, there may be provided twice as many second rotational detent elements 136 as first rotational detent elements 122. Alternatively, in other embodiments, there may be provided twice as many first rotational detent elements 122 as second rotational detent elements 136.

In certain preferred embodiments, such as those shown throughout the several drawings, the releasable engagement may be adapted to be in male-female fashion. In particular such embodiments, the first rotational detent elements 122 may be of the female form and the second rotational detent elements 136 may be of the male form. Alternatively, in other embodiments (not shown), the first rotational detent elements 122 may be of the male form and the second rotational detent elements 136 may be of the female form.

Certain embodiments may further comprise an ostomy pouch 102 having an internal fluid reservoir 104. In such embodiments, the proximal portion 112 may be mated to the ostomy pouch 102 such that the first bore 118 is in fluid communication with the internal fluid reservoir 104. Such a mating may be accomplish, for example, by way of adhesive bond, RF weld, or the like.

Referring to FIGS. 3 and 4, for example, in particular embodiments, the second valve element 124 may include a second flange element 140 disposed at the upper end 126. The second flange element 140 may have a second flange face 142 being directed generally axially outwardly from the upper portion 130. In such embodiments, for example, the second rotational detent elements 136 may be distributed along the second flange face 142. The second valve element 124 may further include a plurality of griping elements 150 radially distributed about the upper portion 130, each of which may intersect the second flange element 140.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A fluid discharge valve for releasing fluid from a urostomy pouch, said fluid discharge valve comprising:
a first valve element including a proximal end, a distal end and a first bore extending therebetween, a proximal portion adjacent said proximal end, a distal portion adjacent said distal end, and a first flange element disposed generally between said proximal and distal portions, said first flange element including a multiplicity of radially-distributed first rotational detent elements; and
a second valve element including an upper end, a lower end, a second bore extending therebetween, an upper portion adjacent said upper end, and a lower portion adjacent said lower end, said upper portion being adapted to threadedly receive at least a segment of said distal portion for threaded movement of said second valve element axially with respect to said first valve element between an open configuration and a closed configuration, said upper end including a plurality of radially distributed second rotational detent elements, said second rotational detent elements being adapted to come into releasable engagement with respective said first rotational detent elements when said second valve element is moved into its closed configuration;
wherein said first bore is sealed shut when said second valve element is in its closed configuration, and said releasable engagement inhibits threaded movement of said second valve element out of its closed configuration.

2. A fluid discharge valve as defined in claim 1 in which said inhibition is adapted to be overcome by application of a greater rotational force on said second valve element than is required to effectuate substantially the remainder of said threaded movement.

3. A fluid discharge valve as defined in claim 1 in which said first valve element includes a first axial detent element and said second valve element includes a second axial detent element, said first and second axial detent elements being adapted to engage one another to prevent said second valve element from axially separating from said first valve element when said second valve element is in its open configuration.

4. A fluid discharge valve as defined in claim 1 in which said first flange element includes a first flange face, said first flange face being generally axially directed toward said distal portion, said first rotational detent elements being distributed along said first flange face.

5. A fluid discharge valve as defined in claim 1 in which said first and second rotational detent elements are radially distributed in a substantially even fashion.

6. A fluid discharge valve as defined in claim 1 in which there are twice as many second rotational detent elements as first rotational detent elements.

7. A fluid discharge valve as defined in claim 1 in which there are twice as many first rotational detent elements as second rotational detent elements.

8. A fluid discharge valve as defined in claim 1 in which said releasable engagement is adapted to be in male-female fashion, wherein said first rotational detent elements are of the female form and said second rotational detent elements are of the male form.

9. A fluid discharge valve as defined in claim 1 in which said releasable engagement is adapted to be in male-female fashion, wherein said first rotational detent elements are of the male form and said second rotational detent elements are of the female form.

10. A fluid discharge valve as defined in claim 1 in which said second valve element includes a flow conditioner within said second bore, said flow conditioner having a plug element and one or more vanes extending radially outwardly thereof, said first bore being adapted to be sealed shut by said plug element when said second valve element is in its closed configuration.

11. A fluid discharge valve as defined in claim 1 further comprising an ostomy pouch having an internal fluid reservoir, said proximal portion being mated to said ostomy pouch such that said first bore is in fluid communication with said internal fluid reservoir.

12. A fluid discharge valve for releasing fluid from a urostomy pouch, said fluid discharge valve comprising:
a first valve element including a proximal end, a distal end and a first bore extending therebetween, a proximal portion adjacent said proximal end, a distal portion adjacent said distal end, a first axial detent element, and a first flange element disposed generally between said proximal and distal portions, said first flange element includes a first flange face and a multiplicity of radially-distributed first rotational detent elements, said first flange face being generally axially directed toward said distal portion, said first rotational detent elements being distributed along said first flange face; and
a second valve element including an upper end, a lower end, a second bore extending therebetween, an upper portion adjacent said upper end, a lower portion adjacent said lower end, a second axial detent element, and a flow conditioner within said second bore, said flow conditioner having a plug element and one or more vanes extending radially outwardly thereof, said upper portion being adapted to threadedly receive at least a segment of said distal portion for threaded movement of said second valve element axially with respect to said first valve element between an open configuration and a closed configuration, said upper end including a plurality of radially distributed second rotational detent elements, said second rotational detent elements being adapted to come into releasable engagement with respective said first rotational detent elements when said second valve element is moved into its closed configuration;
wherein said first bore is sealed shut by said plug element when said second valve element is in its closed configuration, said releasable engagement inhibits threaded movement of said second valve element out of its closed configuration, and said first and second axial detent elements are adapted to engage one another to prevent said second valve element from axially separating from said first valve element when said second valve element is in its open configuration.

13. A fluid discharge valve as defined in claim 12 in which said inhibition is adapted to be overcome by application of a greater rotational force on said second valve element than is generally required to effectuate the remainder of said threaded movement.

14. A fluid discharge valve as defined in claim 12 in which said first and second rotational detent elements are radially distributed in a substantially even fashion.

15. A fluid discharge valve as defined in claim 12 in which said releasable engagement is adapted to be in male-female fashion, wherein said first rotational detent elements are of the female form and said second rotational detent elements are of the male form.

16. A fluid discharge valve as defined in claim 12 in which said releasable engagement is adapted to be in male-female fashion, wherein said first rotational detent elements are of the male form and said second rotational detent elements are of the female form.

17. A fluid discharge valve as defined in claim 12 in which said second valve element includes a second flange element disposed at said upper end, said second flange element having a second flange face being directed generally axially outwardly from said upper portion, said second rotational detent elements being distributed along said second flange face.

18. A fluid discharge valve as defined in claim 12 further comprising an ostomy pouch having an internal fluid reservoir, said proximal portion being mated to said ostomy pouch such that said first bore is in fluid communication with said internal fluid reservoir.

19. A fluid discharge valve as defined in claim 1 in which:
(a) said threaded movement requires a first torque throughout a majority of said movement between said closed and open configurations;
(b) an initial release of said second valve element from its closed configuration requires a second torque; and
(c) return of said second valve element into its closed configuration requires a third torque;
wherein said second and third torques are greater than said first torque.

20. A fluid discharge valve for releasing fluid from a urostomy pouch, said fluid discharge valve comprising:
a first valve element including a proximal end, a distal end and a first bore extending therebetween, a proximal portion adjacent said proximal end, a distal portion adjacent said distal end, a first axial detent element, and a first flange element disposed generally between said proximal and distal portions, said first flange element includes a first flange face and at least one first rotational detent element, said first flange face being generally axially directed toward said distal portion, said at least one first rotational detent element being disposed at said first flange face, said proximal portion being mated to an ostomy pouch having an internal fluid reservoir such that said first bore is in fluid communication with said internal fluid reservoir; and
a second valve element including an upper end, a lower end, a second bore extending therebetween, an upper portion adjacent said upper end, a lower portion adjacent said lower end, a second axial detent element, and a flow conditioner within said second bore, said flow conditioner having a plug element and one or more vanes extending radially outwardly thereof, said upper portion being adapted to threadedly receive at least a segment of said distal portion for threaded movement of said second valve element axially with respect to said first valve element between an open configuration and a closed configuration, said upper end including at least a one second rotational detent element, said at least one second rotational detent element being adapted to come into releasable engagement with respective said at least one first rotational detent element when said second valve element is moved into its closed configuration;
wherein said first bore is sealed shut by said plug element when said second valve element is in its closed configuration, said releasable engagement inhibits threaded movement of said second valve element out of its closed configuration, said inhibition being adapted to be overcome by application of a greater rotational force on said second valve element than is required to effectuate substantially the remainder of said threaded movement, and said first and second axial detent elements are adapted to engage one another to prevent said second valve element from axially separating from said first valve element when said second valve element is in its open configuration.
